(19) United States
(12) Patent Application Publication
(10) Pub. No.: US 2025/0204918 A1
(43) Pub. Date: Jun. 26, 2025

(54) OCCLUSIVE IMPLANT SYSTEMS

(71) Applicant: Boston Scientific Scimed, Inc., Maple Grove, MN (US)

(72) Inventors: Patrick Willoughby, Shoreview, MN (US); Scott Andrew McCullough, Plymouth, MN (US); Emily Vance, Minneapolis, MN (US)

(73) Assignee: Boston Scientific Scimed, Inc., Maple Grove, MN (US)

(21) Appl. No.: 18/987,148

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00

(52) U.S. Cl. CPC A61B 17/12122; A61B 17/12031; A61B 17/12159; A61B 17/12172; A61B 2017/00477; A61B 2017/00862; A61B 2017/00867; A61B 2017/1205

(57) ABSTRACT

An occlusive implant system includes an occlusive implant comprising an expandable framework, and an occlusive disk configured to be fixedly attached to the occlusive implant. An occlusive portion of the disk may be disposed entirely proximal of the expandable framework. An occlusive implant system may include a core wire having a lumen, and an elongate strand slidably disposed within the lumen, wherein a distal end of the strand is secured to the framework. The core wire may be configured to advance the occlusive disk over the elongate strand and attach the occlusive disk to the occlusive implant. A kit for use with an occlusive implant system may include a plurality of occlusive disks. Each occlusive disk may be configured to be coupled to the expandable framework. Each occlusive disk may have a maximum radial extent in an unconstrained configuration different from other occlusive disks.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

In one example, an occlusive implant system for occluding a left atrial appendage may comprise an occlusive implant comprising an expandable framework configured to shift between a collapsed configuration and a deployed configuration, and an occlusive disk configured to be fixedly attached to a proximal portion of the occlusive implant. An occlusive portion of the occlusive disk may be disposed entirely proximal of the expandable framework when the occlusive disk is fixedly attached to the proximal portion of the occlusive implant.

In addition or alternatively to any example described herein, the occlusive portion of the occlusive disk comprises an expandable foam component configured to shift from a delivery configuration to an expanded configuration in vivo.

In addition or alternatively to any example described herein, the expandable foam component comprises a shape memory foam.

In addition or alternatively to any example described herein, the expandable foam component is configured to adapt and conform to surrounding anatomy when shifting from the delivery configuration to the expanded configuration.

In addition or alternatively to any example described herein, the occlusive disk comprises a distal extension configured to be fixedly attached to a proximal hub of the occlusive implant.

In addition or alternatively to any example described herein, the expandable foam component comprises an axial passageway that is open in the delivery configuration and closed in the expanded configuration.

In addition or alternatively to any example described herein, an occlusive implant system for occluding a left atrial appendage may comprise a core wire having a lumen extending therethrough, an occlusive implant releasably couplable to a distal end of the core wire, the occlusive implant comprising an expandable framework configured to shift between a collapsed configuration and a deployed configuration, an occlusive disk configured to be attached to a proximal portion of the occlusive implant, and an elongate strand slidably disposed within the lumen of the core wire. A distal end of the elongate strand may be secured to the expandable framework. The core wire may be configured to advance the occlusive disk over the elongate strand and attach the occlusive disk to the proximal portion of the occlusive implant.

In addition or alternatively to any example described herein, the core wire is configured to advance the occlusive disk over the elongate strand to the occlusive implant and attach the occlusive disk to the proximal portion of the occlusive implant after shifting the expandable framework to the deployed configuration.

In addition or alternatively to any example described herein, the occlusive disk comprises an expandable foam component configured to shift from a delivery configuration to an expanded configuration in vivo, wherein the expandable foam component is configured to be advanced over the elongate strand to the occlusive implant in the delivery configuration.

In addition or alternatively to any example described herein, the expandable foam component is configured to shift from the delivery configuration to the expanded configuration after attaching the occlusive disk to the occlusive implant.

In addition or alternatively to any example described herein, an occlusive portion of the occlusive disk is disposed entirely proximal of the expandable framework when the occlusive disk is attached to the proximal portion of the occlusive implant.

In addition or alternatively to any example described herein, the occlusive disk is configured to be non-removably attached to the proximal portion of the occlusive implant.

In addition or alternatively to any example described herein, a kit for use with an occlusive implant system may comprise a plurality of occlusive disks. Each occlusive disk of the plurality of occlusive disks may be configured to shift from a delivery configuration toward an expanded configuration in vivo. Each occlusive disk of the plurality of occlusive disks may be configured to be coupled to an expandable framework of an occlusive implant. Each occlusive disk of the plurality of occlusive disks may have a maximum radial extent in an unconstrained configuration. The maximum radial extent of each occlusive disk of the plurality of occlusive disks may be different from other occlusive disks of the plurality of occlusive disks.

In addition or alternatively to any example described herein, each occlusive disk of the plurality of occlusive disks is configured to be non-removably coupled to the expandable framework of the occlusive implant.

In addition or alternatively to any example described herein, each occlusive disk of the plurality of occlusive disks is disposed entirely proximal of the expandable framework of the occlusive implant after being coupled to the expandable framework of the occlusive implant.

In addition or alternatively to any example described herein, each occlusive disk of the plurality of occlusive disks comprises an expandable foam component configured to shift from a delivery configuration to an expanded configuration in vivo.

In addition or alternatively to any example described herein, each occlusive disk of the plurality of occlusive disks is configured to be coupled to the expandable framework of the occlusive implant prior to implantation of the occlusive implant within a left atrial appendage.

In addition or alternatively to any example described herein, each occlusive disk of the plurality of occlusive disks is configured to be coupled to the expandable framework of the occlusive implant after implantation of the occlusive implant within a left atrial appendage.

In addition or alternatively to any example described herein, each occlusive disk of the plurality of occlusive disks is attachable to a distal end of one core wire.

In addition or alternatively to any example described herein, the distal end of the one core wire is releasably couplable to the occlusive implant.
The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIGS. 1-2 are side views illustrating selected aspects of an occlusive implant system for occluding a left atrial appendage;

FIGS. 3-4 illustrate selected aspects of an occlusive implant system for occluding a left atrial appendage;

FIGS. 5-7 illustrate selected aspects of an occlusive implant system for occluding a left atrial appendage;

FIGS. 8-13 illustrates selected aspects of an occlusive implant system for occluding a left atrial appendage;

FIG. 14 illustrates selected aspects of an alternative configuration of an occlusive implant system for occluding a left atrial appendage;

FIG. 15 illustrates selected aspects of an alternative configuration of an occlusive implant system for occluding a left atrial appendage;

FIG. 16 illustrates selected aspects of an occlusive implant system disposed within a left atrial appendage;

FIG. 17 illustrates selected aspects of an occlusive implant system disposed within a left atrial appendage;

FIG. 18 illustrates selected aspects of an occlusive implant system disposed within a left atrial appendage; and

FIG. 19 illustrates selected aspects of an occlusive implant system disposed within a left atrial appendage.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

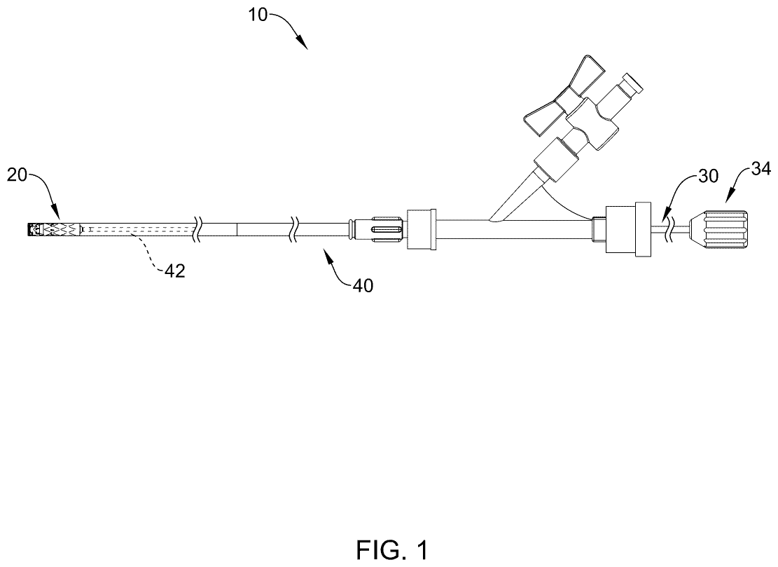

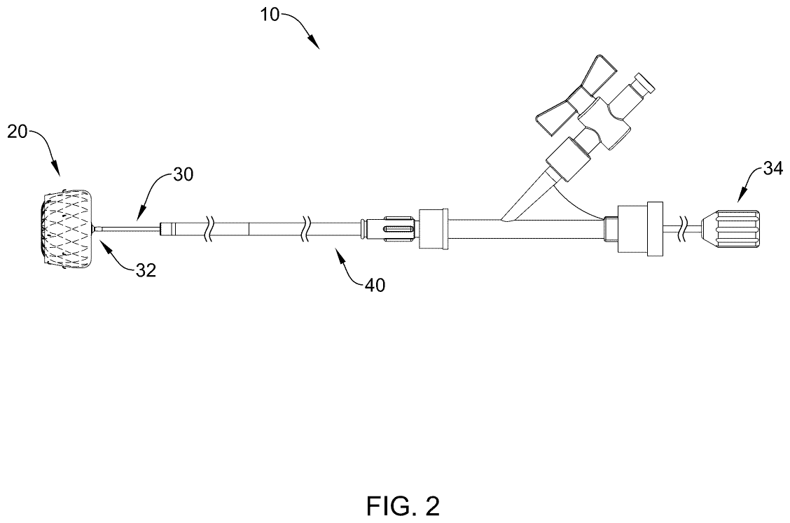

FIGS. 1-2 illustrate selected components and/or arrangements of an occlusive implant system 10 for occluding a left atrial appendage. It should be noted that in any given figure, some features of the occlusive implant system 10 may not be shown, or may be shown schematically, for simplicity. Additional details regarding some of the components of the occlusive implant system 10 may be illustrated in other figures in greater detail. The occlusive implant system 10 may be used to percutaneously deliver and/or deploy a variety of medical implants (e.g., a cardiovascular implant, an occlusive implant, a replacement heart valve implant, etc.) to one or more locations within the anatomy, including but not limited to, in some embodiments, the heart.

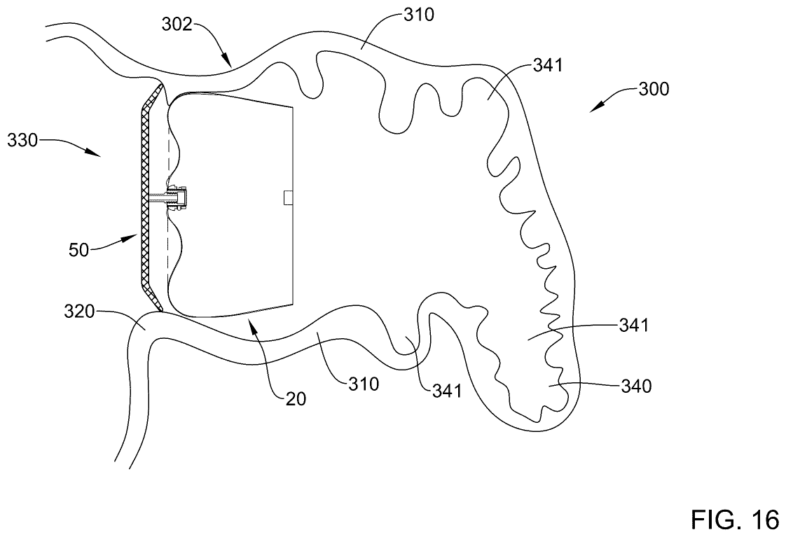

The occlusive implant system 10 may comprise a core wire 30. The occlusive implant system 10 may comprise an occlusive implant 20 releasably couplable to and/or disposable at a distal end 32 of the core wire 30. In at least some embodiments, the occlusive implant 20 may be configured to occlude the left atrial appendage (e.g., ref. 300, FIGS. 16-19). The left atrial appendage is attached to and in fluid communication with the left atrium of the patient's heart. The left atrial appendage may have a complex geometry and/or irregular surface area. In some patients, the left atrial appendage may have a plurality of lobes and/or recesses extending in different directions.

In some embodiments, the occlusive implant system 10 may include a delivery sheath 40 having a lumen 42 (e.g., FIG. 1) extending from a proximal opening to a distal opening. In some embodiments, the core wire 30 may be slidably disposed within the lumen 42 of the delivery sheath 40. The core wire 30 may have a proximal end 34 disposed proximal of the delivery sheath 40. In some embodiments, the proximal end 34 of the core wire 30 may include a knob and/or a handle configured to manipulate and/or move the core wire 30 and/or the occlusive implant 20. In some embodiments, the proximal end 34 of the core wire 30 may include a knob and/or a handle configured to manipulate and/or move the core wire 30 and/or the occlusive implant 20 relative to the delivery sheath 40. In some embodiments, the delivery sheath 40 may be sized and configured to deliver the occlusive implant 20 to the left atrial appendage.

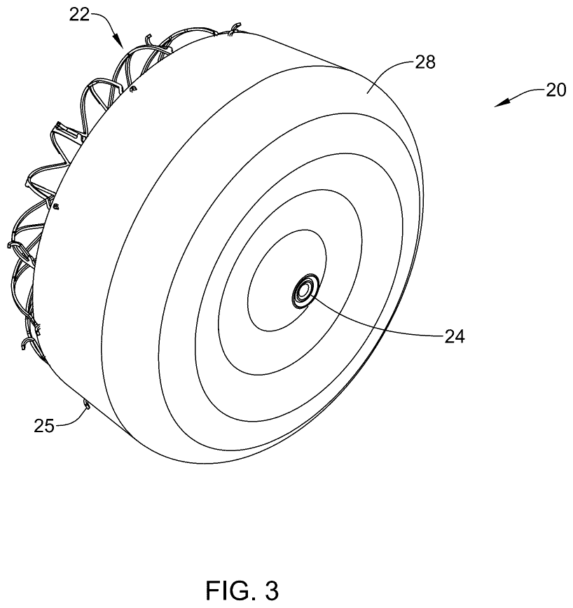

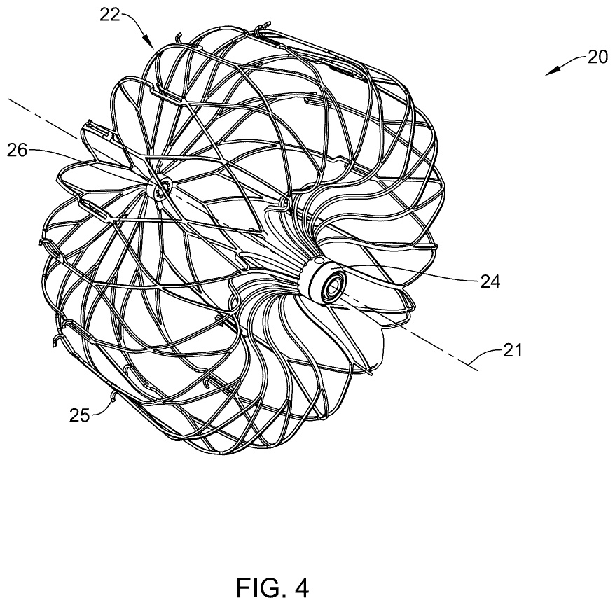

The occlusive implant 20 may include an expandable framework 22 (e.g., FIGS. 3-4) configured to shift between a collapsed configuration (e.g., FIG. 1) and a deployed configuration (e.g., FIGS. 2-4). In some embodiments, the occlusive implant 20 may be disposed within the lumen 42 of the delivery sheath 40 proximate the distal opening in the collapsed configuration. In some embodiments, the delivery sheath 40 may constrain the occlusive implant 20 and/or the expandable framework 22 in the collapsed configuration. In some embodiments, the occlusive implant 20 and/or the expandable framework 22 may be configured to shift from the collapsed configuration to the deployed configuration when the occlusive implant 20 is disposed distal of the distal opening of the lumen 42 and/or the delivery sheath 40, and/or when the occlusive implant 20 is unconstrained. In some embodiments, the occlusive implant 20 and/or the expandable framework 22 may be configured to shift from the collapsed configuration to the deployed configuration when the occlusive implant 20 is unconstrained by the delivery sheath 40. In at least some embodiments, the expandable framework 22 may be self-biased toward the deployed configuration.

In some embodiments, the core wire 30 may be slidably and/or rotatably disposed within the lumen 42 of the delivery sheath 40. In some embodiments, the proximal end 34 of the core wire 30 may extend proximally of a proximal end of the delivery sheath 40 and/or the proximal opening of the lumen 42 for manual manipulation by a clinician or practitioner. In some embodiments, the occlusive implant 20 may be removably attachable, joinable, securable, or otherwise connectable to the distal end 32 of the core wire 30. The core wire 30 may be configured to and/or may be capable of axially translating the occlusive implant 20 relative to the delivery sheath 40. The delivery sheath 40 and/or the core wire 30 may have a selected level of axial stiffness and/or pushability characteristics while also having a selected level of flexibility to permit navigation through the patient's vasculature.

Some suitable, but non-limiting, examples of materials for the occlusive implant system 10, the core wire 30, the delivery sheath 40, and/or the occlusive implant 20, etc. are discussed below.

Turning now to FIGS. 3 and 4, the occlusive implant 20 may comprise an expandable framework 22 configured to shift along a longitudinal axis 21 (e.g., FIG. 4) between the collapsed configuration and the deployed configuration. In the collapsed configuration, the expandable framework 22 may be axially elongated and/or radially compressed. In the deployed configuration, the expandable framework 22 may be axially shortened and/or radially expanded. The expandable framework 22 may comprise a plurality of interconnected struts defining a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements. In some embodiments, the plurality of interconnected struts may converge, join, and/or connect at intersections or nodes.

The plurality of interconnected struts may be formed and/or cut from a tubular member. In some embodiments, the plurality of interconnected struts may be integrally formed and/or cut from a unitary member. In some embodiments, the plurality of interconnected struts may be integrally formed and/or cut from a unitary tubular member and subsequently formed and/or heat set to a desired shape in the deployed configuration. In some embodiments, the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the plurality of interconnected struts include laser cutting, machining, punching, stamping, electro discharge machining (EDM), chemical dissolution, etc. Other means and/or methods are also contemplated.

In some embodiments, the expandable framework 22 may be compliant and substantially conform to and/or be in sealing engagement with the shape and/or geometry of a wall of the left atrial appendage in the deployed configuration. In some embodiments, the occlusive implant 20 may expand to a size, extent, or shape less than or different from a maximum unconstrained extent, as determined by the surrounding tissue and/or wall of the left atrial appendage. In some embodiments, reducing a thickness of various elements of the expandable framework 22 may increase the flexibility and compliance of the expandable framework 22 and/or the occlusive implant 20, thereby permitting the expandable framework 22 and/or the occlusive implant 20 to conform to the tissue around it, rather than forcing the tissue to conform to the expandable framework 22 and/or the occlusive implant 20. In some embodiments, the expandable framework 22 and/or the occlusive implant 20 may be stronger and/or less compliant, and thus the expandable framework 22 and/or the occlusive implant 20 may force the tissue of the left atrial appendage to conform to the expandable framework 22 and/or the occlusive implant 20 in the deployed configuration. Other configurations are also contemplated.

In some embodiments, the occlusive implant 20 and/or the expandable framework 22 may comprise a plurality of anchoring elements 25. In some embodiments, the plurality of anchoring elements 25 may extend radially outward from the expandable framework 22 in the deployed configuration. In at least some embodiments, the plurality of anchoring elements 25 may be configured to engage with tissue and/or may be configured to secure the occlusive implant 20 and/or the expandable framework 22 to tissue at a target site (e.g., the left atrial appendage, etc.). In some embodiments, the plurality of anchoring elements 25 may be configured to prevent dislodgement and/or ejection of the occlusive implant 20 from the target site and/or the left atrial appendage.

In some embodiments, the occlusive implant 20 and/or the expandable framework 22 may comprise a proximal hub 24 and/or a distal hub 26. The longitudinal axis 21 of the expandable framework 22 may extend from the proximal hub 24 to the distal hub 26. In at least some embodiments, the proximal hub 24 and/or the distal hub 26 may be centered on and/or coaxial with the longitudinal axis 21. The plurality of interconnected struts may be joined together at and/or fixedly attached to the proximal hub 24 and/or the distal hub 26. In some embodiments, the proximal hub 24 and/or the distal hub 26 may be fixedly attached to the expandable framework 22 and/or the plurality of interconnected struts, such as by welding, adhesive bonding, brazing, soldering, etc. The proximal hub 24 may be configured to releasably connect, couple, and/or attach the occlusive implant 20 and/or the expandable framework 22 to the distal end 32 of the core wire 30 (e.g., FIGS. 1-2). In some embodiments, the proximal hub 24 may include internal threads configured to rotatably and/or threadably engage external threads formed on and/or at the distal end 32 of the core wire 30. Other configurations and/or structures for releasably securing the occlusive implant 20 to the core wire 30, including but not limited to pins, springs, detents, etc., are also contemplated.

In some embodiments, the occlusive implant 20 may optionally include an occlusive covering 28 connected to, disposed on, disposed over, disposed about, and/or disposed radially outward of a proximal portion of the expandable framework 22 and/or the plurality of interconnected struts. In some embodiments, the occlusive covering 28 may be attached to the proximal hub 24 and/or may be attached to the expandable framework 22 at the proximal hub 24. In some embodiments, the occlusive covering 28 may extend radially outward from and/or may extend distally from the proximal hub 24. In some embodiments, the occlusive covering 28 may be attached and/or secured to the expandable framework 22 at a plurality of discrete locations. In some embodiments, one or more anchoring element(s) of the plurality of anchoring elements 25 may extend through the occlusive covering 28. In some embodiments, the one or more anchoring element(s) of the plurality of anchoring elements 25 extending through the occlusive covering 28 may attach and/or secure the occlusive covering 28 to the expandable framework 22.

In some embodiments, the occlusive covering 28 may include a membrane, a fabric, a mesh, a tissue element, or another suitable construction. In some embodiments, the occlusive covering 28 may be porous. In some embodiments, the occlusive covering 28 may be non-porous. In some embodiments, the occlusive covering 28 may be permeable or impermeable to blood and/or other fluids, such as water. In some embodiments, the occlusive covering 28 may be designed, sized, and/or configured to prevent thrombus and/or embolic material from passing out of the left atrial appendage into the left atrium and/or the patient's bloodstream. In some embodiments, the occlusive covering 28 (e.g., the membrane, the fabric, or the tissue element, etc.) promotes endothelization after implantation, thereby effectively and/or permanently removing the target site (e.g., the left atrial appendage, etc.) from the patient's circulatory system. Some suitable, but non-limiting, examples of materials for the occlusive covering 28 are discussed below.

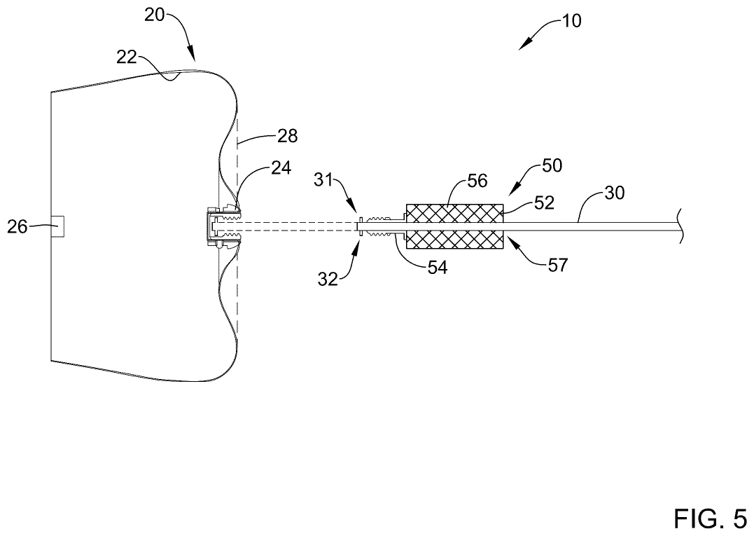

FIGS. 5-7 are partial cross-sectional views illustrating selected aspects of the occlusive implant system 10. In some embodiments, the core wire 30 may be a solid shaft. While not explicitly illustrated, in some embodiments, the core wire 30 may comprise a lumen extending therethrough. Some example configurations of a core wire comprising a lumen are discussed in accordance with other configurations and/or embodiments disclosed herein. In some embodiments, the core wire 30 may be disposed within a hollow sheath (not shown). The core wire 30 may be slidably disposed within the lumen 42 of the delivery sheath 40. In embodiments where the core wire 30 is disposed within a hollow sheath, the hollow sheath may be disposed within the lumen 42 of the delivery sheath 40.

In some embodiments, the core wire 30 may comprise a securing element 31 at and/or proximate the distal end 32 of the core wire 30. In some embodiments, the securing element 31 may be and/or may comprise external threads, a pin or pins, a spring or springs, a detent or detents, etc. In some embodiments, the securing element 31 may be configured to engage the proximal hub 24 of the occlusive implant 20. In some embodiments, the securing element 31 may be configured to engage other structure(s).

In some embodiments, the occlusive implant system 10 may comprise an occlusive disk 50 configured to be attached to a proximal portion of the occlusive implant 20 and/or the expandable framework 22. In some embodiments, the occlusive disk 50 may be configured to be attached to the proximal hub 24. In some embodiments, the occlusive disk 50 may be configured to be fixedly attached and/or non-removably attached to the proximal portion of the occlusive implant 20 and/or the expandable framework 22. In some embodiments, the occlusive disk 50 may be configured to be fixedly attached and/or non-removably attached to the proximal hub 24.

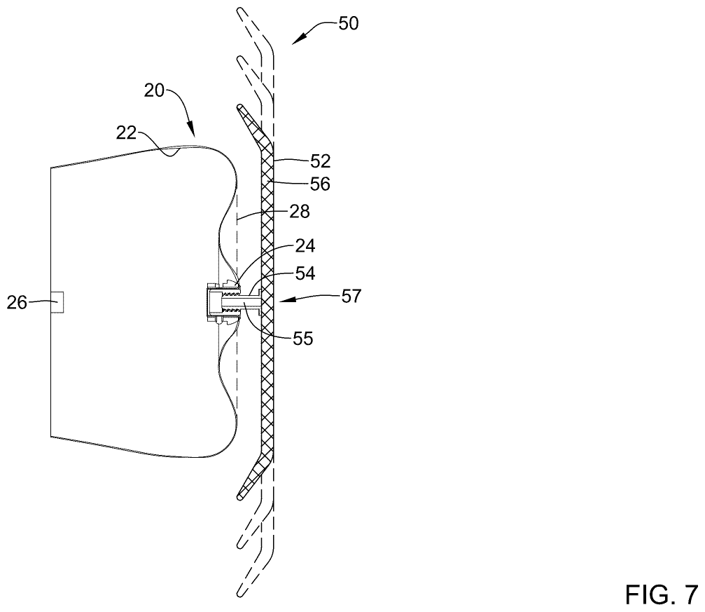

In some embodiments, the occlusive disk 50 may be configured to shift from a delivery configuration toward and/to an expanded configuration in vivo. In some embodiments, the occlusive disk 50 may comprise an occlusive portion 52 (e.g., FIG. 7) and a distal extension 54. In some embodiments, the distal extension 54 may be configured to support at least a portion of the occlusive portion 52. In some embodiments, the distal extension 54 may be fixedly attached to the occlusive portion 52. In some embodiments, the distal extension 54 may be at least partially embedded within the occlusive portion 52. In some embodiments, the distal extension 54 may be non-removably attached to the occlusive portion 52. In some embodiments, the distal extension 54 may be adhesively bonded and/or welded to the occlusive portion 52. In some embodiments, the distal extension 54 may be integrally formed with the occlusive portion 52 as a single monolithic structure. Other configurations are also contemplated.

In some embodiments, the distal extension 54 may comprise an axial lumen 55 (e.g., FIG. 7) extending therethrough configured to receive and/or engage the core wire 30. In some embodiments, the axial lumen 55 of the distal extension 54 may be configured to slidably receive and/or engage the core wire 30. In some embodiments, the distal end 32 of the core wire 30 may be configured to extend and/or slide through the distal extension 54. In some embodiments, the distal extension 54 may be configured to engage the securing element 31 at the distal end 32 of the core wire 30. In some embodiments, the distal extension 54 may be configured to threadably engage the securing element 31 at the distal end 32 of the core wire 30. In some embodiments, the securing element 31 at the distal end 32 of the core wire 30 may be configured to prevent detachment or withdrawal of the core wire 30 from and/or through the distal extension 54 and/or the occlusive disk 50. Other configurations are also contemplated.

In some embodiments, the occlusive portion 52 of the occlusive disk 50 may comprise a generally flattened structure extending radially outward from the distal extension 54 and/or the proximal hub 24 in an unconstrained configuration. In some embodiments, the occlusive portion 52 may comprise a support frame (not shown) coupled to the distal extension 54. In some embodiments, the occlusive disk 50 and/or the occlusive portion 52 may have a maximum radial extent in the unconstrained configuration. In some embodiments, the occlusive disk 50 and/or the occlusive portion 52 and/or the maximum radial extent of the occlusive disk 50 and/or the occlusive portion 52 may be disposed and/or may extend radially outward of the expandable framework 22 in the unconstrained configuration. In some embodiments, a radially outermost perimeter of the occlusive disk 50 and/or the occlusive portion 52 may be disposed and/or may extend radially outward of the expandable framework 22 in the unconstrained configuration.

In some embodiments, the occlusive disk 50 and/or the occlusive portion 52 may include a distal taper along and/or adjacent the radially outermost perimeter thereof in the unconstrained configuration, as seen in FIG. 7. In some embodiments, the occlusive disk 50 and/or the occlusive portion 52 may be devoid of any distal taper along and/or adjacent the radially outermost perimeter thereof in the unconstrained configuration (e.g., the occlusive disk 50 and/or the occlusive portion 52 may be substantially flat, except for a gentle rounding at the radially outermost perimeter, in the unconstrained configuration). In some embodiments, the occlusive disk 50 and/or the occlusive portion 52 may be substantially devoid of sharp corners and/or edges in the unconstrained configuration.

In some embodiments, the occlusive portion 52 of the occlusive disk 50 may comprise an expandable foam component 56 configured to shift from a delivery configuration (e.g., a compressed and/or unexpanded configuration) toward and/or to an expanded configuration. In at least some embodiments, the expandable foam component 56 may be configured to shift from the delivery configuration toward and/or to the expanded configuration in vivo. In some embodiments, the expandable foam component 56 may be configured to shift and/or to bias the occlusive disk 50 and/or the occlusive portion 52 from the delivery configuration toward and/or to the expanded configuration in vivo. In some embodiments, the occlusive portion 52 and/or the expandable foam component 56 may be configured to adapt and conform to surrounding anatomy (e.g., the wall of the left atrial appendage) when shifting from the delivery configuration toward and/or to the expanded configuration.

In some embodiments, the expandable foam component 56 may comprise a shape memory foam. The expandable foam component 56 and/or the shape memory foam may be formed from a biocompatible material. In at least some embodiments, the expandable foam component 56 may be non-biodegradable and/or non-bioabsorbable. In some alternative embodiments, the expandable foam component 56 may be biodegradable and/or bioabsorbable over time. In some embodiments, the expandable foam component 56 may be configured to promote endothelization and/or tissue ingrowth. In some embodiments, the expandable foam component 56 may include a coating, a material, and/or a component that promotes endothelization and/or tissue ingrowth. Other configurations are also contemplated.

In some embodiments, the occlusive portion 52 and/or the expandable foam component 56 may comprise an axial passageway 57 that is open in the delivery configuration to permit the core wire 30 to pass therethrough and closed in the expanded configuration to prevent thrombus or embolic material from passing through the axial passageway 57 (from within the left atrial appendage toward and/or to the left atrium). In at least some embodiments, the axial passageway 57 may be coaxial with the longitudinal axis 21 of the expandable framework 22. In at least some embodiments, the axial passageway 57 may be closed by the expandable foam component 56 shifting toward and/or to the expanded configuration. Other configurations are also contemplated.

In some embodiments, the occlusive portion 52 of the occlusive disk 50 may be disposed entirely proximal of the expandable framework 22 when the occlusive disk 50 is attached to the proximal portion of the occlusive implant 20 and/or the expandable framework 22. In some embodiments, the occlusive portion 52 of the occlusive disk 50 may be disposed entirely proximal of the expandable framework 22 when the occlusive disk 50 is attached to the proximal hub 24. In some embodiments, the occlusive portion 52 of the occlusive disk 50 may be disposed entirely proximal of the expandable framework 22 when the occlusive disk 50 is fixedly attached and/or non-removably attached to the proximal portion of the occlusive implant 20 and/or the expandable framework 22. In some embodiments, the occlusive portion 52 of the occlusive disk 50 may be disposed entirely proximal of the expandable framework 22 when the occlusive disk 50 is fixedly attached and/or non-removably attached to the proximal hub 24.

In some embodiments, the distal extension 54 may be configured to attach the occlusive disk and/or the occlusive portion 52 to the proximal portion of the occlusive implant 20 and/or the expandable framework 22. In some embodiments, the distal extension 54 may be configured to attach the occlusive disk and/or the occlusive portion 52 to the proximal hub 24. In some embodiments, the distal extension 54 may be configured to fixedly attach and/or non-removably attach the occlusive disk and/or the occlusive portion 52 to the proximal portion of the occlusive implant 20 and/or the expandable framework 22. In some embodiments, the distal extension 54 may be configured to fixedly attach and/or non-removably attach the occlusive disk and/or the occlusive portion 52 to the proximal hub 24.

In some embodiments, the distal extension 54 may be configured to space the occlusive portion 52 apart from the expandable framework 22 and/or the proximal hub 24. In some embodiments, the distal extension 54 may be configured to space the occlusive portion 52 proximally from the expandable framework 22 and/or the proximal hub 24.

In some embodiments, the occlusive disk 50 and/or the expandable foam component 56 may be configured to shift from the delivery configuration toward and/or to the expanded configuration after attaching the occlusive disk 50 to the occlusive implant 20. In some embodiments, the occlusive disk 50 and/or the expandable foam component 56 may be configured to shift from the delivery configuration toward and/or to the expanded configuration after fixedly attaching and/or non-removably attaching the occlusive disk 50 to the occlusive implant 20. In some embodiments, the occlusive disk 50 and/or the expandable foam component 56 may be configured to shift from the delivery configuration toward and/or to the expanded configuration after attaching the distal extension 54 to the occlusive implant 20. In some embodiments, the occlusive disk 50 and/or the expandable foam component 56 may be configured to shift from the delivery configuration toward and/or to the expanded configuration after fixedly attaching and/or non-removably attaching the distal extension 54 to the occlusive implant 20. In some embodiments, the occlusive disk 50 and/or the expandable foam component 56 may be configured to shift from the delivery configuration toward and/or to the expanded configuration after attaching the distal extension 54 to the proximal hub 24. In some embodiments, the occlusive disk 50 and/or the expandable foam component 56 may be configured to shift from the delivery configuration toward and/or to the expanded configuration after fixedly attaching and/or non-removably attaching the distal extension 54 to the proximal hub 24.

In some embodiments, a kit for use with an occlusive implant system (e.g., the occlusive implant system 10) may comprise a plurality of occlusive disks. In some embodiments, each occlusive disk of the plurality of occlusive disks may be configured as described herein with respect to the occlusive disk 50 whether or not explicitly described with respect to each occlusive disk of the plurality of occlusive disks. Accordingly, each occlusive disk of the plurality of occlusive disks may comprise at least one, some, or all characteristic(s) of the occlusive disk 50 described herein. In some embodiments, each occlusive disk of the plurality of occlusive disks may comprise different characteristic(s) of the occlusive disk 50. In some embodiments, each occlusive disk of the plurality of occlusive disks may not necessarily comprise the same characteristic(s) as other occlusive disks of the plurality of occlusive disks.

In some embodiments, each occlusive disk of the plurality of occlusive disks may be configured to shift from a delivery configuration toward and/or to an expanded configuration. In some embodiments, each occlusive disk of the plurality of occlusive disks may be configured to shift from the delivery configuration toward and/or to the expanded configuration in vivo.

In some embodiments, each occlusive disk of the plurality of occlusive disks may be configured to be coupled and/or attached to the expandable framework 22 of an occlusive implant (e.g., the occlusive implant 20). In some embodiments, each occlusive disk of the plurality of occlusive disks may be configured to be fixedly coupled and/or fixedly attached to the expandable framework 22 of an occlusive implant (e.g., the occlusive implant 20). In some embodiments, each occlusive disk of the plurality of occlusive disks may be configured to be non-removably coupled and/or non-removably attached to the expandable framework 22 of an occlusive implant (e.g., the occlusive implant 20).

In some embodiments, each occlusive disk of the plurality of occlusive disks may be disposed entirely proximal of the expandable framework 22 of the occlusive implant 20 after being coupled and/or attached to the expandable framework 22 and/or the proximal hub 24 of the occlusive implant 20. In some embodiments, each occlusive disk of the plurality of occlusive disks may be disposed entirely proximal of the expandable framework 22 of the occlusive implant 20 after being fixedly coupled and/or fixedly attached to the expandable framework 22 and/or the proximal hub 24 of the occlusive implant 20. In some embodiments, each occlusive disk of the plurality of occlusive disks may be disposed entirely proximal of the expandable framework 22 of the occlusive implant 20 after being non-removably coupled and/or non-removably attached to the expandable framework 22 and/or the proximal hub 24 of the occlusive implant 20. In some embodiments, each occlusive disk of the plurality of occlusive disks may comprise an expandable foam component 56 configured to shift from a delivery configuration to an expanded configuration in vivo.

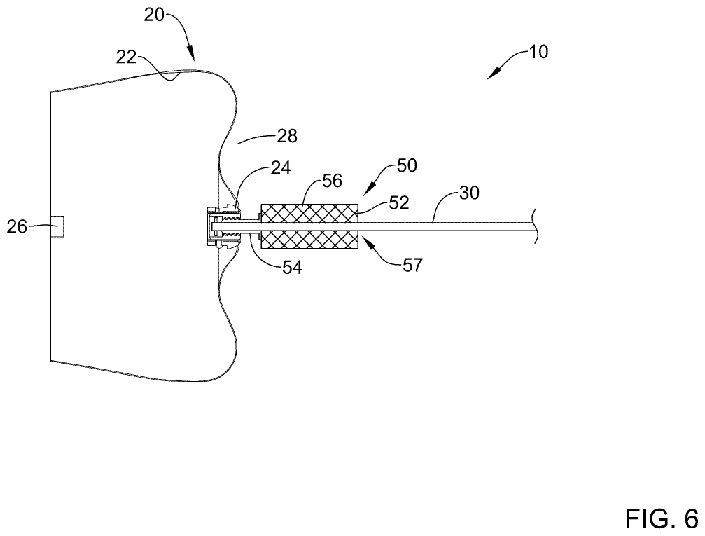

In some embodiments, each occlusive disk of the plurality of occlusive disks may be configured to be coupled and/or attached to the expandable framework 22 of the occlusive implant 20 prior to implantation of the occlusive implant 20 within the left atrial appendage, as seen schematically in FIGS. 5-6. In some embodiments, each occlusive disk of the plurality of occlusive disks may be configured to be fixedly coupled and/or fixedly attached to the expandable framework 22 of the occlusive implant 20 prior to implantation of the occlusive implant 20 within the left atrial appendage. In some embodiments, each occlusive disk of the plurality of occlusive disks may be configured to be non-removably coupled and/or non-removably attached to the expandable framework 22 of the occlusive implant 20 prior to implantation of the occlusive implant 20 within the left atrial appendage.

In use, the practitioner may select an occlusive disk 50 and/or may select one occlusive disk from the plurality of occlusive disks to couple and/or attach to the occlusive implant 20 prior to implantation of the occlusive implant 20 within the left atrial appendage. Selection of the occlusive disk 50 and/or one occlusive disk from the plurality of occlusive disks may depend on one or more factors including but not limited to size, shape, depth, etc. of the left atrial appendage to be occluded.

For the purpose of illustration only, the occlusive implant 20 and/or the expandable framework 22 is shown in FIGS. 5-6 as being in the deployed configuration to show relative positioning of selected features. However, it shall be understood that the expandable framework 22 may be in the collapsed configuration before and/or while the occlusive disk 50 and/or one occlusive disk from the plurality of occlusive disks is being coupled and/or attached to the occlusive implant 20.

As shown in FIG. 5, the occlusive disk 50 and/or one occlusive disk of the plurality of occlusive disks may be advanced over and/or along the core wire 30 to a position adjacent to and/or at the distal end 32 of the core wire 30. In some embodiments, the core wire 30 may be configured to advance the occlusive disk 50 and/or one occlusive disk of the plurality of occlusive disks toward and/or to the proximal portion of the occlusive implant 20 and/or the proximal hub 24.

In some embodiments, the occlusive disk 50 and/or one occlusive disk of the plurality of occlusive disks may be positioned such that the distal extension 54 is engaged with the distal end 32 of the core wire 30 and/or the securing element 31 at and/or proximate the distal end 32 of the core wire 30. Thereafter, the core wire 30 and the occlusive disk 50 and/or one occlusive disk of the plurality of occlusive disks may be advanced toward the proximal portion of the occlusive implant 20 and/or the proximal hub 24 until the occlusive disk 50 and/or one occlusive disk of the plurality of occlusive disks engages the occlusive implant 20 and/or the proximal hub 24. In some embodiments, the core wire 30 and the occlusive disk 50 and/or one occlusive disk of the plurality of occlusive disks may be advanced toward the proximal portion of the occlusive implant 20 and/or the proximal hub 24 until the distal extension 54 engages the occlusive implant 20 and/or the proximal hub 24.

In some embodiments, the distal end 32 of the core wire 30 and/or the securing element 31 at and/or proximate the distal end 32 of the core wire 30 may be disposed within the occlusive implant 20, the expandable framework 22, and/or the proximal hub 24, as shown in phantom in FIG. 5. Thereafter, the occlusive disk 50 and/or one occlusive disk from the plurality of occlusive disks may be advanced over and/or along the core wire 30 until the occlusive disk 50 and/or one occlusive disk from the plurality of occlusive disks engages the occlusive implant 20 and/or the proximal hub 24. In some embodiments, the occlusive disk 50 and/or one occlusive disk from the plurality of occlusive disks may be advanced over and/or along the core wire 30 until the distal extension 54 engages the occlusive implant 20 and/or the proximal hub 24.

Next, the distal extension 54 of the occlusive disk 50 and/or one occlusive disk from the plurality of occlusive disks may be coupled and/or attached to the proximal hub 24 of the occlusive implant 20, as seen in FIG. 6. In some embodiments, the distal extension 54 of the occlusive disk 50 and/or one occlusive disk from the plurality of occlusive disks may be fixedly coupled and/or fixedly attached to the proximal hub 24 of the occlusive implant 20. In some embodiments, the distal extension 54 of the occlusive disk 50 and/or one occlusive disk from the plurality of occlusive disks may be non-removably coupled and/or non-removably attached to the proximal hub 24 of the occlusive implant 20.

As seen in FIG. 6, the expandable foam component 56 of the occlusive disk 50 and/or one occlusive disk from the plurality of occlusive disks may be disposed in the delivery configuration when coupled and/or attached to the proximal hub 24 of the occlusive implant 20.

After coupling and/or attaching the distal extension 54 to the proximal hub 24, the occlusive implant 20 and the occlusive disk 50 (and/or one occlusive disk from the plurality of occlusive disks) may be advanced, inserted, and/or implanted into the left atrial appendage with the occlusive implant 20 in the collapsed configuration and the occlusive disk 50 (and/or one occlusive disk from the plurality of occlusive disks) and/or the expandable foam component 56 in the delivery configuration. Once disposed within the left atrial appendage, the occlusive implant 20 may be shifted toward and/or to the deployed configuration. Once disposed within the left atrial appendage, the occlusive disk 50 (and/or one occlusive disk from the plurality of occlusive disks) and/or the expandable foam component 56 may be shifted toward and/or to the expanded configuration, in vivo. As the expandable foam component 56 expands and/or shifts toward the expanded configuration, the expandable foam component 56 and/or the occlusive disk 50 (and/or one occlusive disk from the plurality of occlusive disks) may conform to the surrounding anatomy and/or tissues (e.g., the wall of the left atrial appendage, the ostium of the left atrial appendage, etc.) to seal off the left atrial appendage.

After shifting the occlusive implant 20 toward and/or to the deployed configuration and after shifting the occlusive disk 50 (and/or one occlusive disk from the plurality of occlusive disks) and/or the expandable foam component 56 toward and/or to the expanded configuration in vivo, the core wire 30 may be disengaged, decoupled, and/or detached from the occlusive implant 20, the proximal hub 24, the occlusive disk 50, and/or one occlusive disk from the plurality of occlusive disks. In some embodiments, after coupling and/or attaching the distal extension 54 to the proximal hub 24, the core wire 30 may be removed and/or withdrawn proximally from the occlusive implant 20, the proximal hub 24, the occlusive disk 50, and/or one occlusive disk from the plurality of occlusive disks.

Upon removing and/or withdrawing proximally the core wire 30 from the occlusive implant 20, the proximal hub 24, the occlusive disk 50, and/or one occlusive disk from the plurality of occlusive disks, the expandable foam component 56 may expand radially inward to close the axial passageway 57 and/or the axial lumen 55, as seen in FIG. 7 (wherein the anatomy and/or the left atrial appendage is omitted from the figure).

In some embodiments, each occlusive disk of the plurality of occlusive disks may have a maximum radial extent in an unconstrained configuration. In at least some embodiments, the maximum radial extent of each occlusive disk of the plurality of occlusive disks may be different from other occlusive disks of the plurality of occlusive disks (e.g., each occlusive disk may have a different maximum radial extent), as seen in phantom in FIG. 7. In some embodiments, each occlusive disk of the plurality of occlusive disks may have a maximum radial diameter in an unconstrained configuration. In at least some embodiments, the maximum radial diameter of each occlusive disk of the plurality of occlusive disks may be different from other occlusive disks of the plurality of occlusive disks (e.g., each occlusive disk may have a different maximum radial diameter). In some embodiments, each occlusive disk of the plurality of occlusive disks may have an outer perimeter length (e.g., a circumference) in an unconstrained configuration. In at least some embodiments, the outer perimeter length (e.g., the circumference) of each occlusive disk of the plurality of occlusive disks may be different from other occlusive disks of the plurality of occlusive disks (e.g., each occlusive disk may have a different outer perimeter length or circumference). Other configurations are also contemplated.

In some embodiments, each occlusive disk of the plurality of occlusive disks may have a maximum thickness in an unconstrained configuration and/or in the expanded configuration. In at least some embodiments, the maximum thickness of each occlusive disk of the plurality of occlusive disks may be different from other occlusive disks of the plurality of occlusive disks (e.g., each occlusive disk may have a different maximum thickness). Other configurations and/or characteristics, including combinations thereof, are also contemplated.

While the occlusive disk 50 and/or the plurality of occlusive disks of the kit shown in FIGS. 5-7 are illustrated in conjunction with the occlusive implant 20 of FIGS. 1-4, it is contemplated that the kit and/or the plurality of occlusive disks thereof may be used in conjunction with other occlusive implants of various types and/or shapes. In some embodiments, the means of coupling and/or attaching the occlusive disk 50 and/or the plurality of occlusive disks to the occlusive implant may be modified or changed according to and/or to accommodate the particular occlusive implant being used.

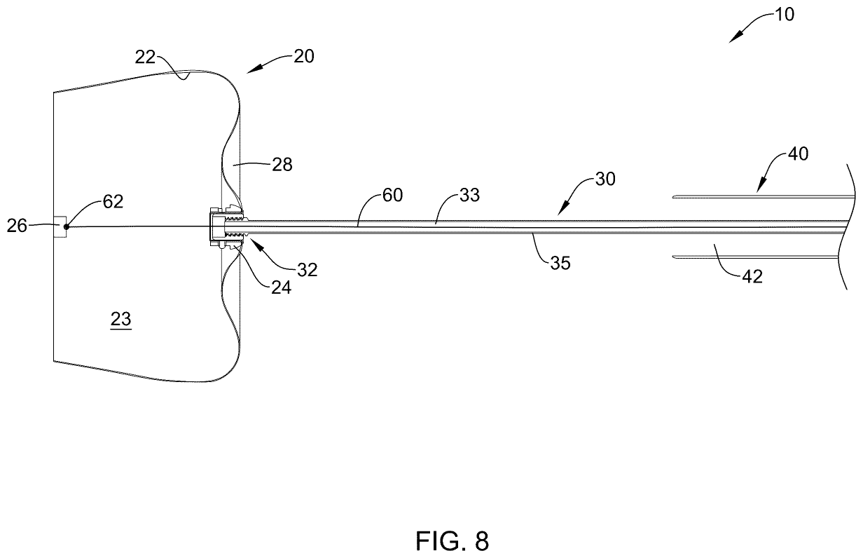

FIGS. 8-10 are partial cross-sectional views illustrating selected aspects of the occlusive implant system 10. For the purpose of illustration, the occlusive implant 20 is shown in the deployed configuration, such as immediately after the occlusive implant 20 has been advanced into, implanted within, and/or deployed within the left atrial implant. In some embodiments, the occlusive implant system 10 may comprise the core wire 30 having a lumen 33 extending therethrough. The core wire 30 may comprise a wall 35 defining the lumen 33. In some embodiments, the wall 35 may extend from the proximal end 34 (e.g., FIGS. 1-2) to the distal end 32 of the core wire 30. In some embodiments, the lumen 33 may extend from the proximal end 34 (e.g., FIGS. 1-2) to the distal end 32 of the core wire 30. Other configurations are also contemplated.

In some embodiments, the core wire 30 may be formed as a tubular member. In some embodiments, the core wire 30 and/or the wall 35 may comprise a single-layered tube. In some embodiments, the core wire 30 and/or the wall 35 may comprise a multi-layered tube. In some embodiments, the core wire 30 and/or the wall 35 may comprise an outer jacket and a tubular coil disposed therein and/or embedded within the outer jacket. In some embodiments, the core wire 30 and/or the wall 35 may comprise an outer jacket and a tubular braid disposed therein and/or embedded within the outer jacket. Other configurations are also contemplated. In at least some embodiments, the core wire 30 and/or the wall 35 may be formed from a polymeric material. In some embodiments, the core wire 30 and/or the wall 35 may be formed from a metallic material. In some embodiments, the core wire 30 and/or the wall 35 may be formed from a combination of metallic and polymeric materials.

In some embodiments, the occlusive implant 20 may be releasably couplable to the distal end 32 of the core wire 30. In at least some embodiments, the distal end 32 of the core wire 30 may include external threads formed on and/or at the distal end 32 of the core wire 30, wherein the external threads are configured to rotatably and/or threadably engage the internal threads of the proximal hub 24. Other configurations are also contemplated.

In some embodiments, the occlusive implant system 10 may comprise an elongate strand 60 slidably disposed within the lumen 33 of the core wire 30. In some embodiments, the elongate strand 60 may comprise a suture, a filament, a wire, a thread, etc. In some embodiments, the elongate strand 60 may have a length that is fixed. In at least some embodiments, the length of the elongate strand 60 may be greater than a length of the core wire 30. In some embodiments, the elongate strand 60 may extend proximal of the proximal end 34 (e.g., FIGS. 1-2) of the core wire 30. In some embodiments, the elongate strand 60 may extend distal of the distal end 32 of the core wire 30. In some embodiments, the elongate strand 60 may extend proximal of the proximal end 34 (e.g., FIGS. 1-2) of the core wire 30 and distal of the distal end 32 of the core wire 30. In some embodiments, the core wire 30 may be rotatable relative to the elongate strand 60.

In some embodiments, the length of the elongate strand 60 may not change under load. In at least some embodiments, the elongate strand 60 may have minimal stretch or elongation. For example, as an axial force is applied to the elongate strand 60, the elongate strand 60 may resist and/or avoid stretching or elongating. In some embodiments, the elongate strand 60 may be axially inelastic. Alternatively, in some embodiments, the elongate strand 60 may be axially elastic and/or may be capable of elongating or stretching by a predetermined amount. Other configurations are also contemplated.

In some embodiments, the elongate strand 60 may extend within the lumen 33 of the core wire 30 to the occlusive implant 20 and/or the expandable framework 22. In some embodiments, the elongate strand 60 may extend alongside the core wire 30 for a first portion of its length and within the lumen 33 of the core wire 30 for a second portion of its length. Other configurations are also contemplated.

In at least some embodiments, the elongate strand 60 may extend into an interior 23 of the occlusive implant 20 and/or the expandable framework 22 in the deployed configuration. In some embodiments, a distal end 62 of the elongate strand 60 may be secured to, coupled to, and/or attached to the expandable framework 22 and/or the occlusive implant 20. In some embodiments, the distal end 62 of the elongate strand 60 may be fixedly attached to the expandable framework 22 and/or the occlusive implant 20. In some embodiments, the distal end 62 of the elongate strand 60 may be secured to, coupled to, and/or attached to the distal hub 26 of the expandable framework 22 and/or the occlusive implant 20, as seen in FIG. 8. In some embodiments, the distal end 62 of the elongate strand 60 may be fixedly attached to the distal hub 26 of the expandable framework 22 and/or the occlusive implant 20.

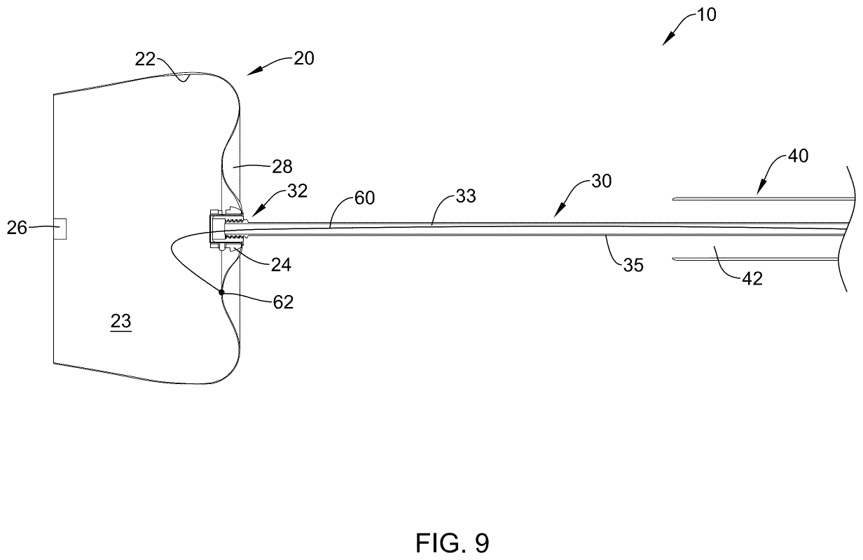

In some embodiments, the distal end 62 of the elongate strand 60 may be secured to, coupled to, and/or attached to the expandable framework 22 of the occlusive implant 20, as seen in FIG. 9. In some embodiments, the distal end 62 of the elongate strand 60 may be secured to, coupled to, and/or attached to a proximal portion of the expandable framework 22 and/or the occlusive implant 20. In some embodiments, the distal end 62 of the elongate strand 60 may be fixedly attached to the proximal portion of the expandable framework 22 and/or the occlusive implant 20.

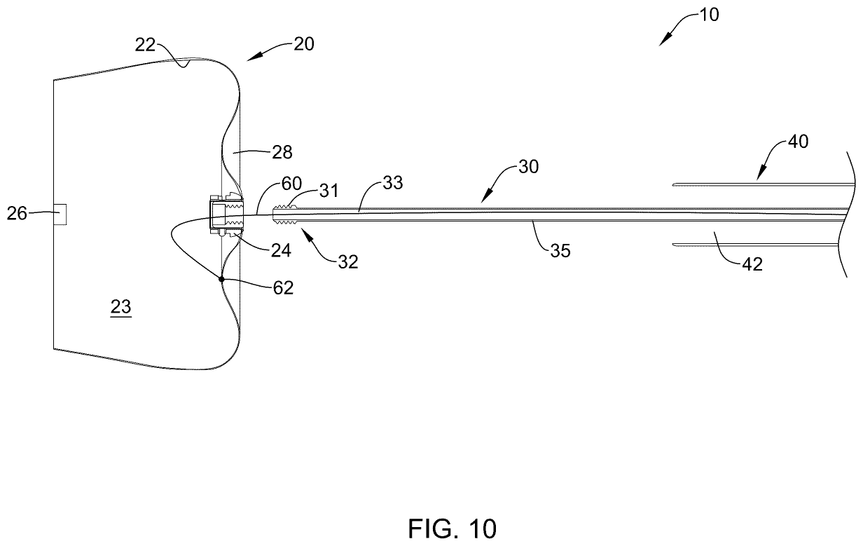

In some embodiments, the elongate strand 60 may be configured to remain coupled to the expandable framework 22 after the core wire 30 has been decoupled from the expandable framework 22 and/or the proximal hub 24, as seen in FIG. 10. This may also apply if and/or when the elongate strand 60 is coupled to the distal hub 26. For the purpose of illustration only, FIG. 10 is shown with the elongate strand 60 coupled to the expandable framework 22. However, the skilled person will recognize that the elongate strand 60 may be configured to remain coupled to the distal hub 26 after the core wire 30 has been decoupled from the expandable framework 22 and/or the proximal hub 24. In at least some embodiments, if a practitioner then determines that the occlusive implant 20 needs to be repositioned, the elongate strand 60 may act as a guide for recoupling the core wire 30 to the proximal hub 24 and/or the expandable framework 22.

In some embodiments, the elongate strand 60 may be unattached to the expandable framework 22. Instead, the elongate strand 60 may be configured to pass through the expandable framework 22 to form a distal loop, wherein both and/or opposing free ends of the elongate strand 60 may extend to a position outside of the core wire 30 and/or the delivery sheath 40. In at least some embodiments, the elongate strand 60 may extend through the core wire 30. In some embodiments, after the core wire 30 has been decoupled from the expandable framework 22 and/or the proximal hub 24, if a practitioner then determines that the occlusive implant 20 needs to be repositioned, the elongate strand 60 may act as a guide for recoupling the core wire 30 to the proximal hub 24 and/or the expandable framework 22.

In some embodiments, after decoupling and/or detaching the core wire 30 from the expandable framework 22 and/or the proximal hub 24, the core wire 30 may be removed from the patient and thereafter, the occlusive disk 50 and/or one occlusive disk of the plurality of occlusive disks may be releasably coupled and/or releasably attached to the distal end 32 of the core wire 30. In at least some embodiments, the occlusive disk 50, one occlusive disk of the plurality of occlusive disks, and/or each occlusive disk of the plurality of occlusive disks may comprise a proximal extension 58 extending proximally therefrom.

In some embodiments, the proximal extension 58 may be fixedly attached to the occlusive portion 52. In some embodiments, the proximal extension 58 may be at least partially embedded within the occlusive portion 52. In some embodiments, the proximal extension 58 may be non-removably attached to the occlusive portion 52. In some embodiments, the proximal extension 58 may be adhesively bonded and/or welded to the occlusive portion 52. In some embodiments, the proximal extension 58 may be integrally formed with the occlusive portion 52 as a single monolithic structure. Other configurations are also contemplated.

In some embodiments, the proximal extension 58 may comprise an axial lumen extending therethrough configured to receive and/or engage the core wire 30. In some embodiments, the axial lumen of the proximal extension 58 may be configured to slidably receive and/or engage the core wire 30. In some embodiments, the distal end 32 of the core wire 30 may be configured to extend and/or slide through the proximal extension 58. In some embodiments, the proximal extension 58 may be configured to engage the securing element 31 at the distal end 32 of the core wire 30. In some embodiments, the proximal extension 58 may be configured to threadably engage the securing element 31 at the distal end 32 of the core wire 30. Other configurations are also contemplated.

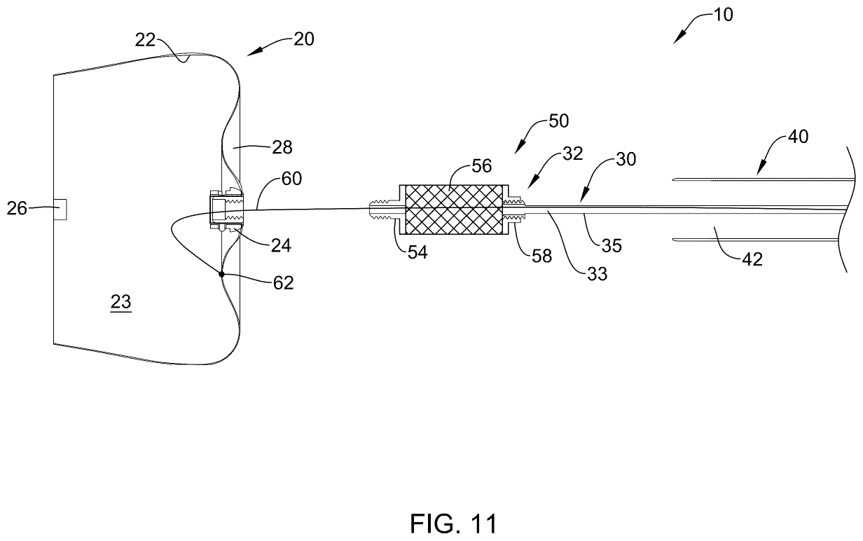

The core wire 30 may be configured to advance the occlusive disk 50 and/or one occlusive disk of the plurality of occlusive disks over and/or on the elongate strand 60 toward and/or to the occlusive implant 20 after shifting the expandable framework 22 toward and/or to the deployed configuration, as seen in FIG. 11. As discussed herein, the occlusive disk 50 and/or one occlusive disk of the plurality of occlusive disks may comprise the expandable foam component 56. In at least some embodiments, the expandable foam component 56 may be configured to be advanced over and/or on the elongate strand 60 to the occlusive implant 20 and/or one occlusive disk of the plurality of occlusive disks in the delivery configuration.

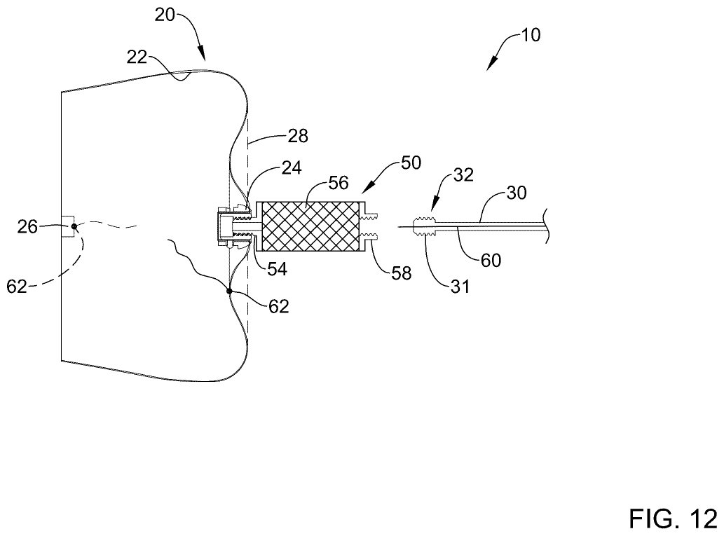

In some embodiments, each occlusive disk of the plurality of occlusive disks may be configured to be coupled and/or attached to the expandable framework 22 of the occlusive implant 20 after implantation of the occlusive implant 20 within the left atrial appendage, as seen in FIG. 12. In some embodiments, each occlusive disk of the plurality of occlusive disks may be configured to be fixedly coupled and/or fixedly attached to the expandable framework 22 of the occlusive implant 20 after implantation of the occlusive implant 20 within the left atrial appendage. In some embodiments, each occlusive disk of the plurality of occlusive disks may be configured to be non-removably coupled and/or non-removably attached to the expandable framework 22 of the occlusive implant 20 after implantation of the occlusive implant 20 within the left atrial appendage.

In at least some embodiments, the core wire 30 may be configured to couple and/or attach the occlusive disk 50 and/or the one occlusive disk of the plurality of occlusive disks to the proximal portion of the occlusive implant 20 and/or the proximal hub 24 in vivo. In some embodiments, the core wire 30 may be configured to fixedly couple and/or fixedly attach the occlusive disk 50 and/or the one occlusive disk of the plurality of occlusive disks to the proximal portion of the occlusive implant 20 and/or the proximal hub 24 in vivo. In at least some embodiments, the core wire 30 may be configured to non-releasably couple and/or non-releasably attach the occlusive disk 50 and/or the one occlusive disk of the plurality of occlusive disks to the proximal portion of the occlusive implant 20 and/or the proximal hub 24 in vivo.

After coupling and/or attaching the occlusive disk 50 and/or the one occlusive disk of the plurality of occlusive disks to the proximal portion of the occlusive implant and/or the proximal hub 24, the distal end 32 and/or the securing element 31 of the core wire 30 may be decoupled and/or detached from the occlusive disk 50 and/or the one occlusive disk of the plurality of occlusive disks, as seen in FIG. 12. In some embodiments, after coupling and/or attaching the occlusive disk 50 and/or the one occlusive disk of the plurality of occlusive disks to the proximal portion of the occlusive implant and/or the proximal hub 24, the distal end 32 and/or the securing element 31 of the core wire 30 may be decoupled and/or detached from the proximal extension 58. In at least some embodiments, after decoupling and/or detaching the core wire 30 from the occlusive disk 50, the one occlusive disk of the plurality of occlusive disks, and/or the proximal extension 58, the occlusive disk 50 and/or the one occlusive disk of the plurality of occlusive disks may remain coupled and/or attached to the occlusive implant 20, the expandable framework 22, and/or the proximal hub 24.

In some embodiments, each occlusive disk of the plurality of occlusive disks may be releasably couplable and/or releasably attachable to a distal end of one core wire. For example, each occlusive disk of the plurality of occlusive disks may have a common coupling or attachment point configured to releasably engage with, releasably couple to, and/or releasably attach to the distal end of one core wire such that each occlusive disk of the plurality of occlusive disks is releasably couplable and/or releasably attachable to the same core wire (at different times—e.g., not simultaneously). In at least some embodiments, the distal end of the one core wire may be releasably couplable and/or releasably attachable to the occlusive implant 20.

After satisfactory deployment of the occlusive implant 20 and coupling and/or attaching the occlusive disk 50 and/or the one occlusive disk of the plurality of occlusive disks thereto, the elongate strand 60 may be decoupled and/or detached from the expandable framework 22 and/or the distal hub 26 (e.g., as shown in phantom), severed along its length, and/or removed from the interior 23 of the occlusive implant 20. In some embodiments, the elongate strand 60 may be decoupled from the expandable framework 22/or the distal hub 26 (e.g., as shown in phantom), severed along its length, and/or removed from the interior 23 of the occlusive implant 20 prior to decoupling and/or detaching the core wire 30 from the occlusive disk 50, the one occlusive disk of the plurality of occlusive disks, and/or the proximal extension 58.

In some embodiments, the elongate strand 60 may be decoupled and/or detached from the expandable framework 22 and/or the distal hub 26 (e.g., as shown in phantom), severed along its length, and/or removed from the interior 23 of the occlusive implant 20 after decoupling and/or detaching the core wire 30 from the occlusive disk 50, the one occlusive disk of the plurality of occlusive disks, and/or the proximal extension 58. In some embodiments, the elongate strand 60 may be decoupled and/or detached from the expandable framework 22 and/or the distal hub 26 (e.g., as shown in phantom), severed along its length, and/or removed from the interior 23 of the occlusive implant 20 while and/or simultaneously to decoupling and/or detaching the core wire 30 from the occlusive disk 50, the one occlusive disk of the plurality of occlusive disks, and/or the proximal extension 58.

In embodiments where the elongate strand 60 is unattached to the expandable framework 22, and/or forms a distal loop and/or passes through the expandable framework 22, the elongate strand 60 may be decoupled from the expandable framework 22 and/or removed from the interior 23 of the occlusive implant 20 by pulling on one end of the elongate strand 60 while releasing its other end. The elongate strand 60 may be pulled through the core wire 30 and/or the delivery sheath 40 and removed from the occlusive implant system 10 and/or the patient.

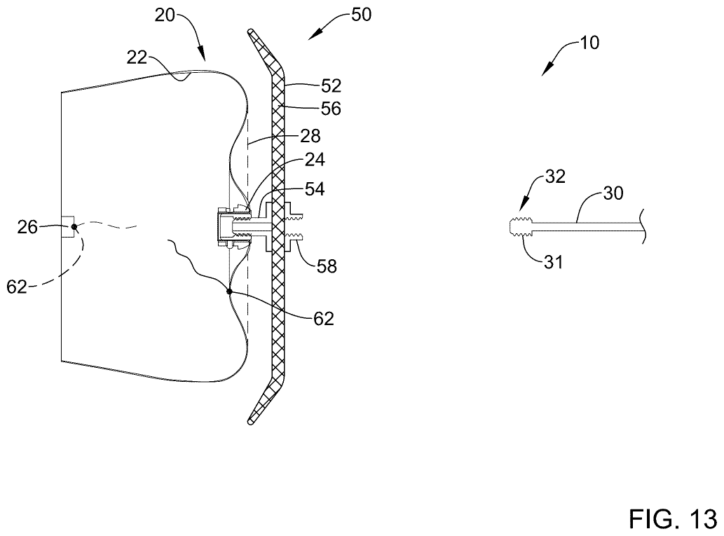

After satisfactory deployment of the occlusive implant 20 and coupling and/or attaching the occlusive disk 50 and/or the one occlusive disk of the plurality of occlusive disks thereto, the occlusive portion 52 of the occlusive disk 50 and/or the one occlusive disk of the plurality of occlusive disks may be configured to shift toward and/or to the expanded configuration in vivo, as seen schematically in FIG. 13. In some embodiments, after satisfactory deployment of the occlusive implant 20 and coupling and/or attaching the occlusive disk 50 and/or the one occlusive disk of the plurality of occlusive disks thereto, the expandable foam component 56 of the occlusive disk 50 and/or the one occlusive disk of the plurality of occlusive disks may be configured to shift toward and/or to the expanded configuration in vivo. In at least some embodiments, the expandable foam component 56 of the occlusive disk 50 and/or the one occlusive disk of the plurality of occlusive disks may be configured to shift toward and/or to the expanded configuration after coupling and/or attaching the occlusive disk 50 and/or the one occlusive disk of the plurality of occlusive disks to the occlusive implant 20, the proximal portion of the occlusive implant 20, and/or the proximal hub 24. In some embodiments, the expandable foam component 56 of the occlusive disk 50 and/or the one occlusive disk of the plurality of occlusive disks may be configured to shift toward and/or to the expanded configuration after coupling and/or attaching the distal extension 54 to the proximal hub 24.

In some embodiments, the expandable foam component 56 of the occlusive disk 50 and/or the one occlusive disk of the plurality of occlusive disks may be configured to shift toward and/or to the expanded configuration after decoupling and/or detaching the core wire 30 from the proximal extension 58. In some embodiments, the expandable foam component 56 of the occlusive disk 50 and/or the one occlusive disk of the plurality of occlusive disks may be configured to shift toward and/or to the expanded configuration before decoupling and/or detaching the core wire 30 from the proximal extension 58. In some embodiments, the expandable foam component 56 of the occlusive disk 50 and/or the one occlusive disk of the plurality of occlusive disks may be configured to shift toward and/or to the expanded configuration simultaneously with decoupling and/or detaching the core wire 30 from the proximal extension 58.

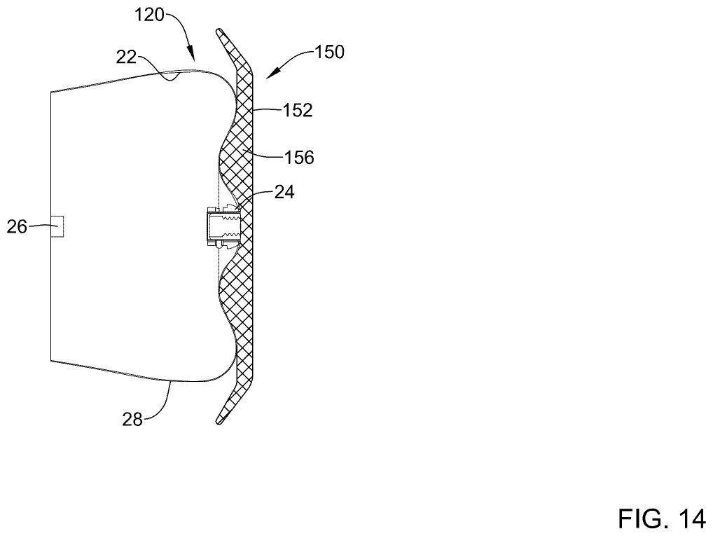

FIG. 14 illustrates selected aspects of an alternative configuration of an occlusive implant 120 of and/or configured to use with the occlusive implant system 10. Physical characteristics and/or structural elements of the occlusive implant 120 may be similar to and/or the same as those described above and/or herein with respect to the occlusive implant 20, except where expressly described otherwise. For example, the occlusive implant 120 may comprise an expandable framework 22, a proximal hub 24, the plurality of anchoring elements 25 (not expressly shown in FIG. 14), a distal hub 26, and/or may optionally comprise an occlusive covering 28, as those features are described herein with respect to the occlusive implant 20.

The occlusive implant 120 may comprise an occlusive disk 150 configured to be attached to a proximal portion of the occlusive implant 20 and/or the expandable framework 22. In some embodiments, the occlusive disk 150 may be configured to be attached to the proximal hub 24. In some embodiments, the occlusive disk 150 may be configured to be fixedly attached and/or non-removably attached to the proximal portion of the occlusive implant 20 and/or the expandable framework 22. In some embodiments, the occlusive disk 50 may be configured to be fixedly attached and/or non-removably attached to the proximal hub 24. In at least some embodiments, the occlusive disk 50 may be fixedly attached and/or non-removably attached directly to the expandable framework 22 and/or the proximal portion of the expandable framework 22. In some embodiments, a proximal facing portion of the expandable framework 22 may be embedded within the occlusive disk 50.

In some embodiments, the occlusive disk 150 may be configured to shift from a delivery configuration toward and/to an expanded configuration in vivo. In some embodiments, the occlusive disk 150 may comprise an occlusive portion 152. In some embodiments, the occlusive portion 152 of the occlusive disk 150 may comprise a generally flattened structure extending radially outward from the proximal hub 24 in an unconstrained configuration. In some embodiments, the occlusive portion 152 may comprise a support frame (not shown) coupled to the proximal hub 24. In some embodiments, the occlusive disk 150 and/or the occlusive portion 152 may have a maximum radial extent in the unconstrained configuration. In some embodiments, the occlusive disk 150 and/or the occlusive portion 152 and/or the maximum radial extent of the occlusive disk 150 and/or the occlusive portion 152 may be disposed and/or may extend radially outward of the expandable framework 22 in the unconstrained configuration. In some embodiments, a radially outermost perimeter of the occlusive disk 150 and/or the occlusive portion 152 may be disposed and/or may extend radially outward of the expandable framework 22 in the unconstrained configuration.

In some embodiments, the occlusive disk 150 and/or the occlusive portion 152 may include a distal taper along and/or adjacent the radially outermost perimeter thereof in the unconstrained configuration. In some embodiments, the occlusive disk 150 and/or the occlusive portion 152 may be devoid of any distal taper along and/or adjacent the radially outermost perimeter thereof in the unconstrained configuration (e.g., the occlusive disk 150 and/or the occlusive portion 152 may be substantially flat, except for a gentle rounding at the radially outermost perimeter, in the unconstrained configuration). In some embodiments, the occlusive disk 150 and/or the occlusive portion 152 may be substantially devoid of sharp corners and/or edges in the unconstrained configuration.

In some embodiments, the occlusive portion 152 of the occlusive disk 150 may comprise an expandable foam component 156 configured to shift from a delivery configuration (e.g., a compressed and/or unexpanded configuration) toward and/or to an expanded configuration. In some embodiments, the proximal facing portion of the expandable framework 22 may be embedded within the expandable foam component 156. In some embodiments, a portion of the occlusive covering 28 (where present) may be embedded within the expandable foam component 156. Other configurations are also contemplated.

In at least some embodiments, the expandable foam component 156 may be configured to shift from the delivery configuration toward and/or to the expanded configuration in vivo. In some embodiments, the expandable foam component 156 may be configured to shift and/or to bias the occlusive disk 150 and/or the occlusive portion 152 from the delivery configuration toward and/or to the expanded configuration in vivo. In some embodiments, the occlusive portion 152 and/or the expandable foam component 156 may be configured to adapt and conform to surrounding anatomy (e.g., the wall of the left atrial appendage) when shifting from the delivery configuration toward and/or to the expanded configuration.

In some embodiments, the expandable foam component 156 may comprise a shape memory foam. The expandable foam component 156 and/or the shape memory foam may be formed from a biocompatible material. In at least some embodiments, the expandable foam component 156 may be non-biodegradable and/or non-bioabsorbable. In some alternative embodiments, the expandable foam component 156 may be biodegradable and/or bioabsorbable over time. In some embodiments, the expandable foam component 156 may be configured to promote endothelization and/or tissue ingrowth. In some embodiments, the expandable foam component 156 may include a coating, a material, and/or a component that promotes endothelization and/or tissue ingrowth. Other configurations are also contemplated.

In some embodiments, the occlusive portion 152 and/or the expandable foam component 156 may comprise an axial passageway that is open in the delivery configuration to permit the core wire 30 to pass therethrough and closed in the expanded configuration to prevent thrombus or embolic material from passing through the axial passageway (from within the left atrial appendage toward and/or to the left atrium). In at least some embodiments, the axial passageway may be coaxial with the longitudinal axis of the expandable framework 22. In at least some embodiments, the axial passageway may be closed by the expandable foam component 156 shifting toward and/or to the expanded configuration. Other configurations are also contemplated.

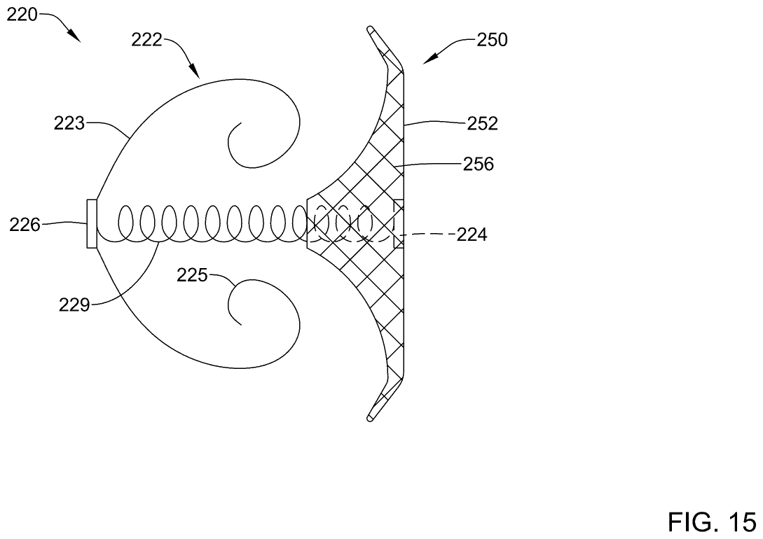

FIG. 15 illustrates selected aspects of an alternative configuration of an occlusive implant 220. In some embodiments, the occlusive implant 220 may comprise an expandable framework 222, a proximal hub 224, the plurality of anchoring elements 225, a distal hub 226, and a spring 229 extending between and/or coupled to the proximal hub 224 and the distal hub 226. The expandable framework 222 may comprise a plurality of arms 223 surrounding the spring 229. In at least some embodiments, the plurality of arms 223 may extend radially outward from the distal hub 226. In some embodiments, the plurality of arms 223 may extend proximally from the distal hub 226. In at least some embodiments, the plurality of anchoring elements 225 may be disposed at free ends of the plurality of arms 223. In some embodiments, the plurality of anchoring elements 225 may be disposed along a length of and/or along a medial portion of the plurality of arms 223 and extend outward therefrom to engage surrounding tissue (e.g., the wall of the left atrial appendage).

The occlusive implant 220 may comprise an occlusive disk 250 coupled to and/or attached to the proximal hub 224 of the occlusive implant 220 and/or the expandable framework 222. In some embodiments, the occlusive disk 250 may be fixedly attached and/or non-removably attached to the proximal hub 224 of the occlusive implant 220 and/or the expandable framework 222. In some embodiments, the proximal hub 224 of the occlusive implant 220 and/or the expandable framework 222 may be embedded within the occlusive disk 250.

In some embodiments, the occlusive disk 250 may be configured to shift from a delivery configuration toward and/to an expanded configuration in vivo. In some embodiments, the occlusive disk 250 may comprise an occlusive portion 252. In some embodiments, the occlusive portion 252 of the occlusive disk 250 may comprise a generally flattened structure extending radially outward from the proximal hub 224 in an unconstrained configuration. In some embodiments, the occlusive portion 252 may comprise a support frame (not shown) coupled to the proximal hub 224. In some embodiments, the occlusive disk 250 and/or the occlusive portion 252 may have a maximum radial extent in the unconstrained configuration. In some embodiments, the occlusive disk 250 and/or the occlusive portion 252 and/or the maximum radial extent of the occlusive disk 250 and/or the occlusive portion 252 may be disposed and/or may extend radially outward of the expandable framework 222 in the unconstrained configuration. In some embodiments, a radially outermost perimeter of the occlusive disk 250 and/or the occlusive portion 252 may be disposed and/or may extend radially outward of the expandable framework 222 in the unconstrained configuration.

In some embodiments, the occlusive disk 250 and/or the occlusive portion 252 may include a distal taper along and/or adjacent the radially outermost perimeter thereof in the unconstrained configuration. In some embodiments, the occlusive disk 250 and/or the occlusive portion 252 may be devoid of any distal taper along and/or adjacent the radially outermost perimeter thereof in the unconstrained configuration (e.g., the occlusive disk 250 and/or the occlusive portion 252 may be substantially flat, except for a gentle rounding at the radially outermost perimeter, in the unconstrained configuration). In some embodiments, the occlusive disk 250 and/or the occlusive portion 252 may be substantially devoid of sharp corners and/or edges in the unconstrained configuration.

In some embodiments, the occlusive portion 252 of the occlusive disk 250 may comprise an expandable foam component 256 configured to shift from a delivery configuration (e.g., a compressed and/or unexpanded configuration) toward and/or to an expanded configuration. In some embodiments, the proximal hub 224 of the occlusive implant 220 and/or the expandable framework 222 may be embedded within the expandable foam component 256.

In at least some embodiments, the expandable foam component 256 may be configured to shift from the delivery configuration toward and/or to the expanded configuration in vivo. In some embodiments, the expandable foam component 256 may be configured to shift and/or to bias the occlusive disk 250 and/or the occlusive portion 252 from the delivery configuration toward and/or to the expanded configuration in vivo. In some embodiments, the occlusive portion 252 and/or the expandable foam component 256 may be configured to adapt and conform to surrounding anatomy (e.g., the wall of the left atrial appendage) when shifting from the delivery configuration toward and/or to the expanded configuration.

In some embodiments, the expandable foam component 256 may comprise a shape memory foam. The expandable foam component 256 and/or the shape memory foam may be formed from a biocompatible material. In at least some embodiments, the expandable foam component 256 may be non-biodegradable and/or non-bioabsorbable. In some alternative embodiments, the expandable foam component 256 may be biodegradable and/or bioabsorbable over time. In some embodiments, the expandable foam component 256 may be configured to promote endothelization and/or tissue ingrowth. In some embodiments, the expandable foam component 256 may include a coating, a material, and/or a component that promotes endothelization and/or tissue ingrowth. Other configurations are also contemplated.

In some embodiments, the occlusive portion 252 of the occlusive disk 250 may be disposed entirely proximal of the expandable framework 222 when the occlusive disk 250 is attached to the proximal hub 224 of the occlusive implant 220 and/or the expandable framework 222. In some embodiments, the occlusive portion 252 of the occlusive disk 50 may be disposed entirely proximal of the expandable framework 222 when the occlusive disk 250 is fixedly attached and/or non-removably attached to the proximal hub 224 of the occlusive implant 220 and/or the expandable framework 222.

FIG. 16 illustrates selected aspects related to implantation of the occlusive implant 20 and the occlusive disk 50, as described with respect to FIGS. 5-7 for example, within a left atrial appendage 300. The left atrial appendage 300 may have a complex geometry and/or irregular surface area. The skilled artisan will recognize that the illustrated left atrial appendage is merely one of many possible shapes and sizes for the left atrial appendage 300, which may vary from patient to patient. The skilled artisan will also recognize that the medical devices and methods disclosed herein may be adapted for various sizes and shapes of the left atrial appendage 300, as necessary. The left atrial appendage 300 may include a generally longitudinal axis arranged along a depth of a main body 302 of the left atrial appendage 300. The main body 302 may include a wall 310 and an ostium 320 forming a proximal mouth 330. In some embodiments, a lateral extent of the ostium 320 and/or the wall 310 may be smaller or less than a depth of the main body 302 along the longitudinal axis, or a depth of the main body 302 may be greater than a lateral extent of the ostium 320 and/or the wall 310. In some embodiments, the left atrial appendage 300 may include a lobe 340 formed in the wall 310. The lobe 340 may be formed or arranged as a tail-like element associated with a distal portion of the main body 302. In some embodiments, the lobe 340 may protrude radially or laterally away from the main body 302. In some embodiments, the left atrial appendage 300 may include a plurality of lobes 341 extending in one or more directions from the main body 302.

As shown in FIG. 16, the occlusive implant 20 may be disposed proximate the ostium 320 and/or the proximal mouth 330 of the left atrial appendage 300. An outer perimeter of the occlusive implant 20 and/or the expandable framework 22 (e.g., FIGS. 5-7) may be engaged with the wall 310 of the left atrial appendage 300. The occlusive disk 50 may be disposed proximal of the occlusive implant 20. The occlusive disk 50 may engage the wall 310 and/or the ostium 320 of the left atrial appendage 300 to further occlude and/or seal off the main body 302 of the left atrial appendage 300. Accordingly, thrombus and/or emboli that form within the left atrial appendage 300 will be trapped therein and prevented from traveling within the patient's vasculature to smaller vessels or sensitive organs.

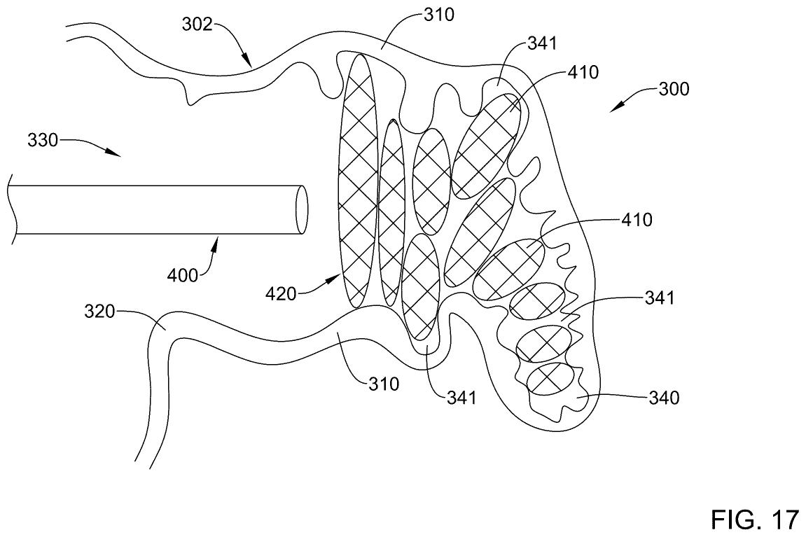

In some embodiments, an occlusive implant system may comprise a delivery device 400 and a plurality of pieces of expandable foam 410 configured to be deployed into the left atrial appendage 300, as seen in FIG. 17. Each piece of the plurality of pieces of expandable foam 410 may be configured to shift from a compressed configuration toward and/or to an expanded configuration. In some embodiments, each piece of the plurality of pieces of expandable foam 410 may be configured to shift from the compressed configuration toward and/or to the expanded configuration when unconstrained by the delivery device 400. In some embodiments, each piece of the plurality of pieces of expandable foam 410 may be configured to shift from the compressed configuration toward and/or to the expanded configuration under predetermined conditions (e.g., at a preselected temperature, when exposed to a preselected fluid or amount of fluid, etc.).

In at least some embodiments, the delivery device 400 may comprise a tubular sheath configured to be advanced to a position adjacent to and/or within the left atrial appendage 300. In some embodiments, the plurality of pieces of expandable foam 410 may be configured to slide in the compressed configuration within the delivery device 400 toward and/or into the left atrial appendage 300. In some embodiments, the plurality of pieces of expandable foam 410 may be configured to slide in the compressed configuration within the delivery device 400 toward and/or into the lobe 340 and/or the plurality of lobes 341 of the left atrial appendage 300. For example, the plurality of pieces of expandable foam 410 may be configured to stack upon each other and/or cooperate to substantially fill the lobe 340 and/or the plurality of lobes 341, as well as at least a portion of the main body 302. The plurality of pieces of expandable foam 410 may be configured to shift toward and/or to the expanded configuration within the lobe 340, the plurality of lobes 341, and/or the main body 302 of the left atrial appendage 300.

In some embodiments, the plurality of pieces of expandable foam 410 may be delivered to and/or advanced into the lobe 340, the plurality of lobes 341, and/or the main body 302 of the left atrial appendage 300 sequentially. In one example, the plurality of pieces of expandable foam 410 may be delivered to and/or advanced into the lobe 340, the plurality of lobes 341, and/or the main body 302 of the left atrial appendage 300 sequentially as first, second, third, etc. pieces of expandable foam. Other configurations and/or examples, including fewer or greater pieces of expandable foam, are also contemplated.

In some embodiments, the plurality of pieces of expandable foam 410 may comprise different sizes and/or shapes in the expanded configuration. For example, some pieces of the plurality of pieces of expandable foam 410 may be oblong, some pieces of the plurality of pieces of expandable foam 410 may be cubic, some pieces of the plurality of pieces of expandable foam 410 may be spherical, some pieces of the plurality of pieces of expandable foam 410 may be flattened, etc. In some embodiments, some pieces of the plurality of pieces of expandable foam 410 may be the same size and/or shape as other pieces of the plurality of pieces of expandable foam 410. In some embodiments, each piece of the plurality of pieces of expandable foam 410 may have a different size and/or shape. Various combinations of size and shape for the plurality of pieces of expandable foam 410 are also contemplated. The use of various sizes and/or shapes for the plurality of pieces of expandable foam 410 may permit the plurality of pieces of expandable foam 410 to be used to substantially fill irregular gaps, spaces, and/or volume within the left atrial appendage 300 with expandable foam while minimizing unfilled space and/or volume within the left atrial appendage 300.

In some embodiments, the occlusive implant system may comprise a plug piece of expandable foam 420. The plug piece of expandable foam 420 may be configured to shift from a compressed configuration toward and/or to an expanded configuration. In some embodiments, the plug piece of expandable foam 420 may be configured to shift from a compressed configuration toward and/or to an expanded configuration when unconstrained by the delivery device 400. In some embodiments, the plug piece of expandable foam 420 may be configured to shift from a compressed configuration toward and/or to an expanded configuration under predetermined conditions (e.g., at a preselected temperature, when exposed to a preselected fluid or amount of fluid, etc.).

In some embodiments, the plug piece of expandable foam 420 may be configured to act as a cap and/or a retainer for the plurality of pieces of expandable foam 410 disposed within the lobe 340, the plurality of lobes 341, and/or the main body 302 of the left atrial appendage 300. For example, the plurality of pieces of expandable foam 410 may be arranged to fill a portion of the lobe 340, the plurality of lobes 341, and/or the main body 302 of the left atrial appendage 300 and then the plug piece of expandable foam 420 may be advanced through the delivery device 400 into the main body 302 of the left atrial appendage 300 and deployed against the plurality of pieces of expandable foam 410. As the plug piece of expandable foam 420 expands, the plug piece of expandable foam 420 may be configured to adapt and conform to the wall 310 of the left atrial appendage 300. In some embodiments, the plug piece of expandable foam 420 may be configured to apply a radially outward force against the wall 310 of the left atrial appendage 300 to secure and/or retain the plug piece of expandable foam 420 within the main body 302 of the left atrial appendage 300. As such, the plug piece of expandable foam 420 will act to hold and/or retain the plurality of pieces of expandable foam 410 in place within the lobe 340, the plurality of lobes 341, and/or the main body 302 of the left atrial appendage 300.

In some embodiments, the plurality of pieces of expandable foam 410 and/or the plug piece of expandable foam 420 may be configured to remain within the lobe 340, the plurality of lobes 341, and/or the main body 302 of the left atrial appendage 300 permanently (e.g., the plurality of pieces of expandable foam 410 and/or the plug piece of expandable foam 420 is never removed from the lobe 340, the plurality of lobes 341, and/or the main body 302 of the left atrial appendage 300 by the practitioner). In some embodiments, the plurality of pieces of expandable foam 410 and/or the plug piece of expandable foam 420 may be configured to be biodegradable over time. In some embodiments, the plurality of pieces of expandable foam 410 and/or the plug piece of expandable foam 420 may be configured to be biodegradable over at least 30 days' time. In some embodiments, the plurality of pieces of expandable foam 410 and/or the plug piece of expandable foam 420 may be configured to be biodegradable over at least 60 days' time. In some embodiments, the plurality of pieces of expandable foam 410 and/or the plug piece of expandable foam 420 may be configured to be biodegradable over at least 90 days' time. In some embodiments, the plurality of pieces of expandable foam 410 and/or the plug piece of expandable foam 420 may be configured to be biodegradable over at least 180 days' time. In some embodiments, the plurality of pieces of expandable foam 410 and/or the plug piece of expandable foam 420 may be configured to be biodegradable over at least 365 days' time. Other configurations are also contemplated.

In some embodiments, the plurality of pieces of expandable foam 410 and/or the plug piece of expandable foam 420 may be configured to prevent thrombus formation (e.g., within the left atrial appendage 300). In some embodiments, the plurality of pieces of expandable foam 410 and/or the plug piece of expandable foam 420 may include anti-thrombus medicament(s). In some embodiments, the plurality of pieces of expandable foam 410 and/or the plug piece of expandable foam 420 may be configured to absorb blood and/or bodily fluid(s). In some embodiments, the plurality of pieces of expandable foam 410 and/or the plug piece of expandable foam 420 may be configured to trap thrombus. In some embodiments, the plurality of pieces of expandable foam 410 and/or the plug piece of expandable foam 420 may be configured to promote tissue ingrowth and/or endothelization. Other configurations are also contemplated.

In at least some embodiments, the plurality of pieces of expandable foam 410 and/or the plug piece of expandable foam 420 may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more. In one example, a piece of expandable foam may have an outer diameter of about 32 millimeters in the expanded configuration and about 3 millimeters in the compressed configuration, producing a collapsibility ratio of at least 10 times (e.g., at least 10:1). Other configurations are also contemplated. In at least some embodiments, the plurality of pieces of expandable foam 410 and/or the plug piece of expandable foam 420 may be configured as open celled foam.

In some embodiments, the plurality of pieces of expandable foam 410 and/or the plug piece of expandable foam 420 may be unconstrained by outside forces and/or structure(s) in the collapsed configuration. In some embodiments, the plurality of pieces of expandable foam 410 and/or the plug piece of expandable foam 420 may be self-maintained in the collapsed configuration by shape memory properties. Other configurations are also contemplated.

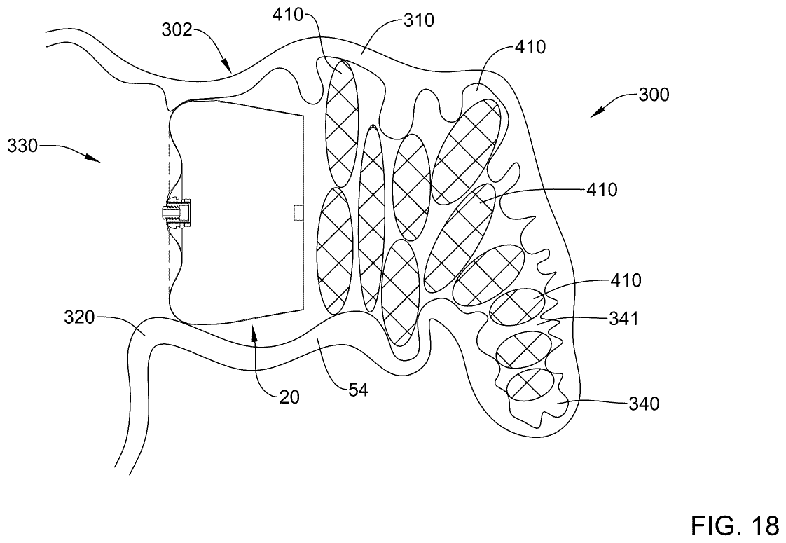

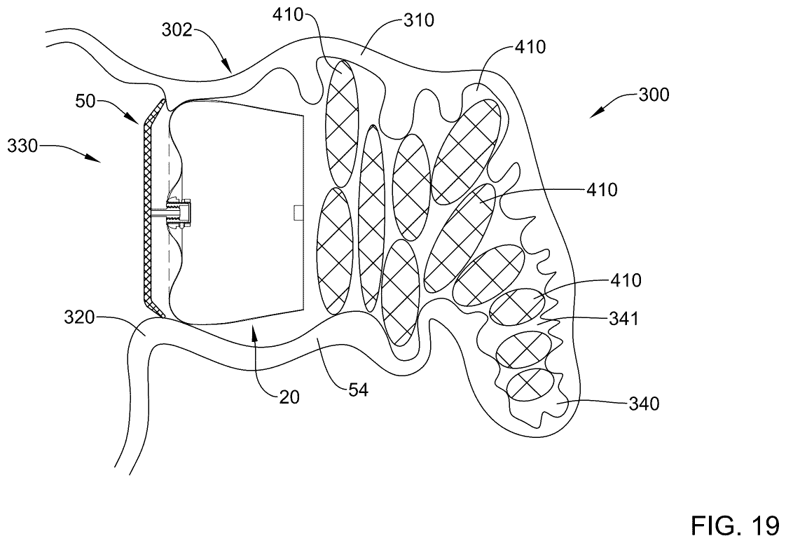

FIGS. 18-19 each illustrate selected aspects of an occlusive implant system as described herein, wherein the occlusive implant system further comprises the plurality of pieces of expandable foam 410 as described with respect to FIG. 17. Similar to the features and/or process described above with respect to FIG. 17, the plurality of pieces of expandable foam 410 may be advanced and/or implanted into the lobe 340, the plurality of lobes 341, and/or the main body 302 of the left atrial appendage 300. However, in contrast to the occlusive implant system of FIG. 17, the occlusive implant system(s) of FIGS. 18-19 utilize the occlusive implant 20 as a plug or retainer for the plurality of pieces of expandable foam 410 instead of the plug piece of expandable foam 420. FIG. 19 further adds the occlusive disk 50 to the occlusive implant 20 shown in FIG. 18. In the occlusive implant system(s) of FIGS. 18-19, the occlusive implant 20 and/or the occlusive disk 50 effectively remove the left atrial appendage 300 from the patient's circulatory system while the plurality of pieces of expandable foam 410 substantially fill the lobe 340, the plurality of lobes 341, and/or the main body 302 of the left atrial appendage 300 to reduce the amount of blood present and/or pooled behind the occlusive implant 20 and thereby further reduce thrombus formation.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the occlusive element, the capsule, the elongate fingers, the elongate strand, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result. In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. An occlusive implant system for occluding a left atrial appendage, comprising:
an occlusive implant comprising an expandable framework configured to shift between a collapsed configuration and a deployed configuration; and
an occlusive disk configured to be fixedly attached to a proximal portion of the occlusive implant;
wherein an occlusive portion of the occlusive disk is disposed entirely proximal of the expandable framework when the occlusive disk is fixedly attached to the proximal portion of the occlusive implant.

2. The occlusive implant system of claim 1, wherein the occlusive portion of the occlusive disk comprises an expandable foam component configured to shift from a delivery configuration to an expanded configuration in vivo.

3. The occlusive implant system of claim 2, wherein the expandable foam component comprises a shape memory foam.

4. The occlusive implant system of claim 2, wherein the expandable foam component is configured to adapt and conform to surrounding anatomy when shifting from the delivery configuration to the expanded configuration.

5. The occlusive implant system of claim 2, wherein the occlusive disk comprises a distal extension configured to be fixedly attached to a proximal hub of the occlusive implant.

6. The occlusive implant system of claim 2, wherein the expandable foam component comprises an axial passageway that is open in the delivery configuration and closed in the expanded configuration.

7. An occlusive implant system for occluding a left atrial appendage, comprising:
a core wire having a lumen extending therethrough;
an occlusive implant releasably couplable to a distal end of the core wire, the occlusive implant comprising an expandable framework configured to shift between a collapsed configuration and a deployed configuration;
an occlusive disk configured to be attached to a proximal portion of the occlusive implant; and
an elongate strand slidably disposed within the lumen of the core wire, wherein a distal end of the elongate strand is secured to the expandable framework;
wherein the core wire is configured to advance the occlusive disk over the elongate strand and attach the occlusive disk to the proximal portion of the occlusive implant.

8. The occlusive implant system of claim 7, wherein the core wire is configured to advance the occlusive disk over the elongate strand to the occlusive implant and attach the occlusive disk to the proximal portion of the occlusive implant after shifting the expandable framework to the deployed configuration.

9. The occlusive implant system of claim 8, wherein the occlusive disk comprises an expandable foam component configured to shift from a delivery configuration to an expanded configuration in vivo, wherein the expandable foam component is configured to be advanced over the elongate strand to the occlusive implant in the delivery configuration.

10. The occlusive implant system of claim 9, wherein the expandable foam component is configured to shift from the delivery configuration to the expanded configuration after attaching the occlusive disk to the occlusive implant.

11. The occlusive implant system of claim 7, wherein an occlusive portion of the occlusive disk is disposed entirely proximal of the expandable framework when the occlusive disk is attached to the proximal portion of the occlusive implant.

12. The occlusive implant system of claim 7, wherein the occlusive disk is configured to be non-removably attached to the proximal portion of the occlusive implant.

13. A kit for use with an occlusive implant system, comprising:
a plurality of occlusive disks, wherein each occlusive disk of the plurality of occlusive disks is configured to shift from a delivery configuration toward an expanded configuration in vivo;
wherein each occlusive disk of the plurality of occlusive disks is configured to be coupled to an expandable framework of an occlusive implant;
wherein each occlusive disk of the plurality of occlusive disks has a maximum radial extent in an unconstrained configuration;
wherein the maximum radial extent of each occlusive disk of the plurality of occlusive disks is different from other occlusive disks of the plurality of occlusive disks.

14. The kit of claim 13, wherein each occlusive disk of the plurality of occlusive disks is configured to be non-removably coupled to the expandable framework of the occlusive implant.

15. The kit of claim 13, wherein each occlusive disk of the plurality of occlusive disks is disposed entirely proximal of the expandable framework of the occlusive implant after being coupled to the expandable framework of the occlusive implant.

16. The kit of claim 13, wherein each occlusive disk of the plurality of occlusive disks comprises an expandable foam component configured to shift from a delivery configuration to an expanded configuration in vivo.

17. The kit of claim 13, wherein each occlusive disk of the plurality of occlusive disks is configured to be coupled to the expandable framework of the occlusive implant prior to implantation of the occlusive implant within a left atrial appendage.

18. The kit of claim 13, wherein each occlusive disk of the plurality of occlusive disks is configured to be coupled to the expandable framework of the occlusive implant after implantation of the occlusive implant within a left atrial appendage.

19. The kit of claim 13, wherein each occlusive disk of the plurality of occlusive disks is attachable to a distal end of one core wire.

20. The kit of claim 19, wherein the distal end of the one core wire is releasably couplable to the occlusive implant.
